(19) United States
(12) Patent Application Publication
(10) Pub. No.: US 2009/0217444 A1
(43) Pub. Date: Sep. 3, 2009

(54) DIVING MASK

(76) Inventor: Chen-Lieh Pan, Yilan Hsien (TW)

Correspondence Address: CHRISTOPHER P. MAIORANA, P.C., 24840 HARPER SUITE 100, ST. CLAIR SHORES, MI, 48080, US

(21) Appl. No.: 12/039,058

(22) Filed: Feb. 28, 2008

(51) Int. Cl. A61F 9/02

(52) U.S. Cl. 2/428; 2/439; 2/452; 2/426

(57) ABSTRACT

A diving mask has a mask portion and a skirt mounted securely to the mask portion. The skirt has an opening with a contact surface and has at least two pads. The contact surface of the opening fits and covers a diver's eyes to prevent water from permeating the mask, when worn. The pads are made of soft and resilient material, are attached to the contact surface of the opening of the skirt and respectively correspond to corners of the diver's eyes. Each pad may be an arch and defines a gap between the pad and the contact surface so when a diver wears the diving mask, the pads closely abut around the corners of the diver's eyes to avoid the corners being pressed. Therefore, the diving mask is ensures a diver experiences normal eyesight and comfort for improved safety.

### BACKGROUND OF THE INVENTION

1. Field of the Invention

The present invention relates to a diving mask, and more particularly to a modified diving mask for improved comfort.

2. Description of Related Art

Diving masks are worn when observing objects or doing activities under water including snorkeling, scuba diving, free diving and spear fishing.

With reference to FIG. 5, a diving mask generally comprises a mask portion (40) and a skirt (50). The mask portion (40) has a solid lens frame and a pair of lenses (41) securely mounted in the lens frame. The skirt (50) is normally made of soft and resilient material and has an opening (51) with a contact surface to fit and cover the eyes and may further cover the nose portion of the face, to create a seal that prevents water from permeating the mask, when worn.

However, the skirt (50) needs to be attached tightly to the diver's face to ensure a seal formed between the skirt (50) and a diver's face when wearing the diving mask. Thus, for divers with wide faces, the contact surface may press against corners of their eyes and make the divers feel uncomfortable, interfere with eyesight and therefore reduce safety of operation.

To overcome the shortcomings, the present invention provides a diving mask for a portable electronic device to obviate or mitigate the aforementioned problems.

### SUMMARY OF THE INVENTION

The main objective of the present invention is to provide a diving mask that does not press corners of a diver's eyes, and ensures a diver experiences normal eyesight and comfort for improved safety.

To achieve the objective, the diving mask in accordance with present invention comprises a mask portion and a skirt.

The skirt is mounted securely with the mask portion, is a soft and resilient material such as plastic, rubber, silicone or the like and has an opening with a contact surface and further has at least two pads. The contact surface of the opening corresponds to and covers the diver's eyes and may further cover a diver's nose to prevent water from permeating the mask. The pads are a soft and resilient material including but not limited to plastic, rubber, silicon or the like, are attached to the contact surface of the opening of the skirt and respectively correspond to corners of the diver's eyes. Each pad may be an arch and defines a gap between the pad and the contact surface.

When the diver wears the diving mask, the pads closely bridge over the corners of the diver's eyes to avoid the eye corners being pressed. Meanwhile, the contact surface of opening of the skirt may still press tightly to a diver's face to ensure a seal is formed between the skirt and the diver's face and secure attachment of the diving mask and therefore, is comfortable and maintains regular vision for improved safety.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a diving mask in accordance with the present invention;

FIG. 2 is an operational side view of the diving mask in FIG. 1;

FIG. 3 is an operational top view of the diving mask in FIG. 1;

FIG. 4 is an enlarged top view in partial section of the modified diving mask in FIG. 1; and

FIG. 5 is a perspective view of a diving mask in accordance with prior art.

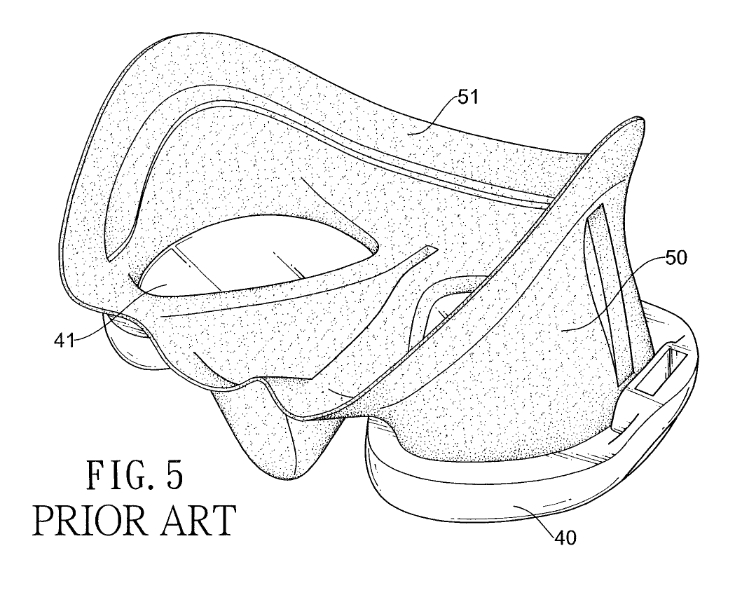

### DETAILED DESCRIPTION OF THE PREFERRED EMBODIMENT

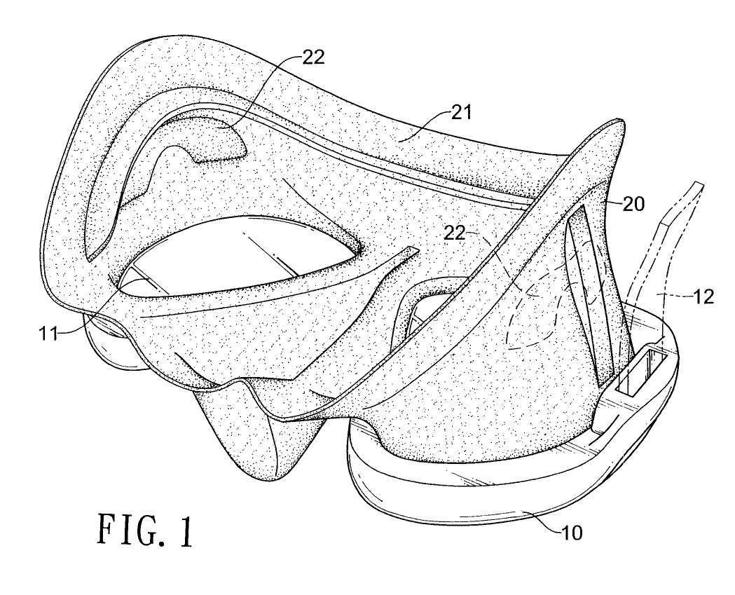

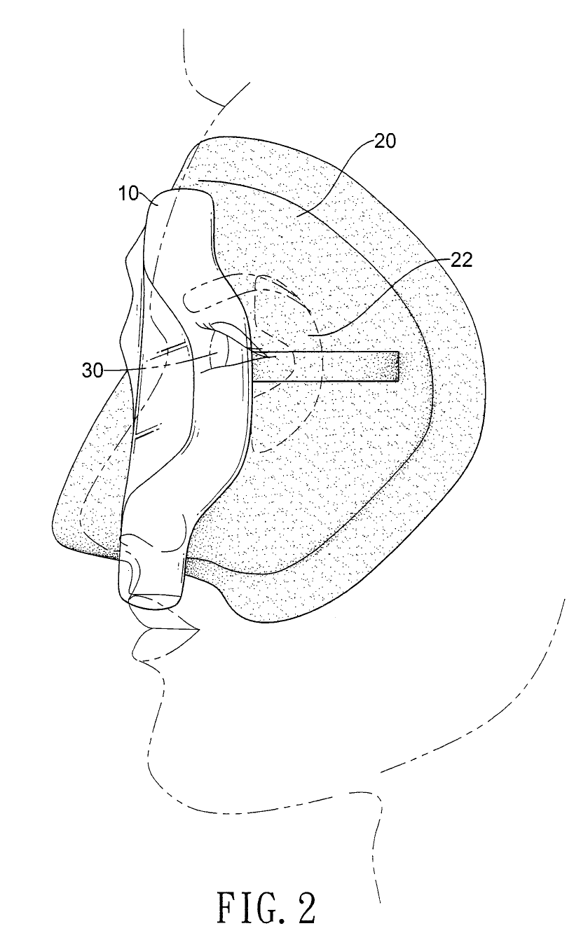

With reference to FIGS. 1 and 2, a diving mask in accordance with present invention comprises a mask portion (10) and a skirt (20).

The mask portion (10) has a solid lens frame, a pair of lenses (11) and a pair of strap mounts (12). The lens frame has two sides. The lenses (11) are securely mounted in the lens frame. The strap mounts (12) are respectively attached to the two sides of the lens frame (10).

The skirt (20) is sealably mounted securely around the mask portion (10), is made of soft and resilient material including but not limited to plastic, rubber, silicon and the like and has an opening (21) with a contact surface and at least two pads (22). The contact surface of the opening (21) corresponds to and cover a diver's eyes (30) to prevent water from permeating the mask when worn and has an optional nose portion corresponding to and covering a diver's nose and communicating with the opening (21) to allow underwater mask clearance. The pads (22) are made of soft and resilient material including, but not limited to plastic, rubber silicon and the like are oppositely attached to the contact surface of the opening (21), may be formed integrally on the contact surface of the skirt (20) or attached thereto and respectively correspond to corners of the diver's eye (30). Each pad (22) is an arch, corresponding to and bridging over the corner of the diver's eyes (30) to define a gap between the pad (22) and the contact surface.

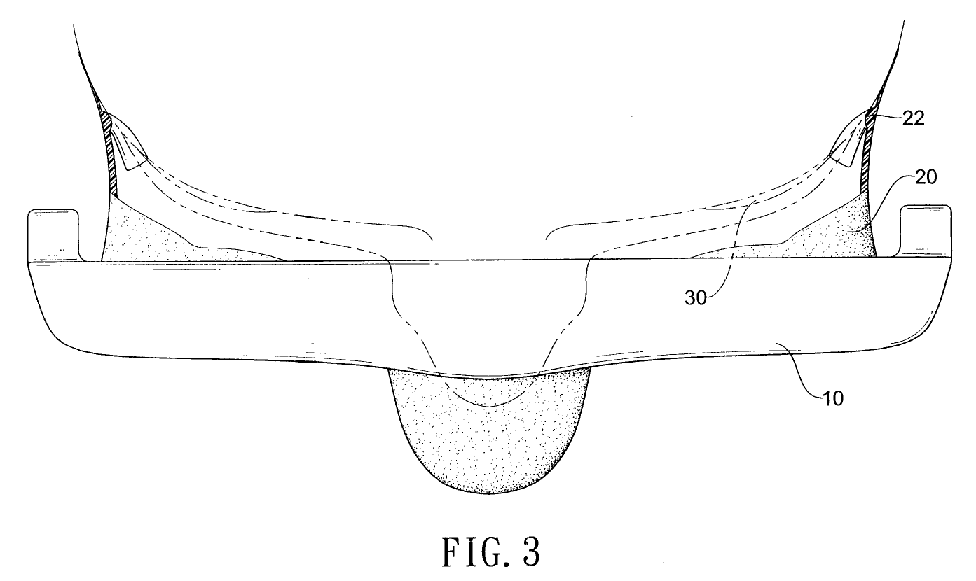

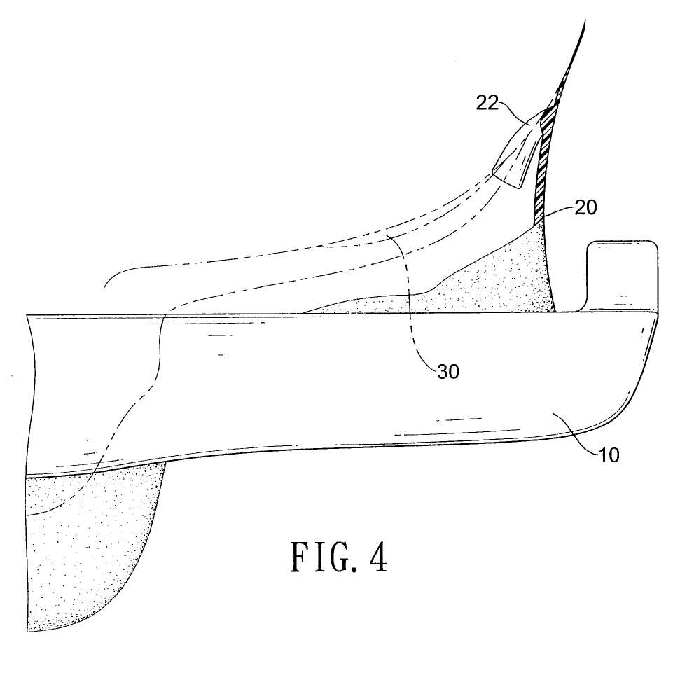

With further reference to FIGS. 3 and 4, when a diver wears the diving mask, the pads (22) closely abut but bridge the corner of the diver's eye (30) to avoid the corners of the diver's eye (30) from being pressed. Meanwhile, the contact surface of opening (21) of the skirt (20) may still be attached tightly to the diver's face to ensure a seal between the skirt (20) and the diver's face and therefore more comfortable and reliable operation.

With in such an arrangement, the diving mask ensures a diver experiences normal eyesight and comfort for improved safety.

Even though numerous characteristics and advantages of the present invention have been set forth in the foregoing description together with details of the structure and function of the invention, the disclosure is illustrative only. Changes may be made in detail especially in matters of shape, size and arrangement of parts within the principles of the invention to the full extent indicated by the broad general meaning of the terms in which the appended claims are expressed.

What is claimed is:

1. A diving mask comprising
a mask portion having
a solid lens frame; and
a pair of lenses being securely mounted in the lens frame; and
a skirt being mounted securely with the mask portion, being made of soft and resilient material and having
an opening with a contact surface; and
at least two pads being soft and resilient and being formed oppositely on the contact surface of the opening of the skirt, each pad defining a gap between the pad and the contact surface.

2. The diving mask as claimed in claim 1, wherein each pad is an arch.

3. The diving mask as claimed in claim 1, wherein the mask portion further has a pair of strap mounts being mounted on two sides of the lens frame.
